(19) United States
(12) Patent Application Publication
(10) Pub. No.: US 2023/0330120 A1
(43) Pub. Date: Oct. 19, 2023

(54) COMPOSITION FOR PREVENTING HAIR LOSS OR PROMOTING HAIR REGROWTH

(71) Applicant: LG HOUSEHOLD & HEALTH CARE LTD., Seoul (KR)

(72) Inventors: Jae-Young SHIN, Seoul (KR); Jae-Yoon KIM, Seoul (KR); Yun-Ho CHOI, Seoul (KR); Sanghwa LEE, Seoul (KR)

(73) Assignee: LG HOUSEHOLD & HEALTH CARE LTD., Seoul (KR)

(21) Appl. No.: 18/042,719

(22) PCT Filed: Aug. 25, 2020
(86) PCT No.: PCT/KR2020/011318

§ 371 Date: Feb. 23, 2023

(51) Int. Cl. A61K 31/7048; A61K 31/22; A61K 31/343; A61K 31/352; A61K 31/575; A61K 31/7034; A61K 31/191; A61P 17/14; A61K 31/704

(52) U.S. Cl. CPC A61K 31/7048; A61K 31/22; A61K 31/343; A61K 31/352; A61K 31/575; A61K 31/7034; A61K 31/191; A61P 17/14; A61K 31/704

(57) ABSTRACT

The present invention relates to a composition for preventing (alleviating) hair loss or promoting hair regrowth, comprising an ingredient for promoting hair regrowth or retarding hair loss. In this regard, in-vitro experiments were conducted in the present invention to verify the effects of promoting the wnt/beta-catenin signaling pathway activity, proliferating dermal papilla cells, and increasing an expression level of beta-catenin protein. Furthermore, the composition was found to have a high effect of preventing hair loss and/or promoting hair regrowth because a better result was obtained as assayed by researchers' evaluation through clinical photography and by phototrichogram after application thereof. In light of such comprehensive outcomes, the composition of the present invention has an excellent effect of preventing hair loss and/or promoting hair regrowth.

### TECHNICAL FIELD

The present disclosure relates to a composition for promoting hair regrowth or preventing (alleviating) hair loss, which contains an ingredient which induces promotion of hair regrowth or prevention of hair loss.

### BACKGROUND ART

With the increasing interest in beauty day by day, hair accounts for an important part. Hair is a thin, keratinized structure produced on the surface of skin. It acts as a cushion against external impact, protects the human body from external stimuli such as direct sunlight, cold, friction, etc. and discharges heavy metals harmful to the body, such as arsenic, mercury, zinc, etc. Recently, the aspect of beauty is also being emphasized.

However, an increasing number of people suffer from hair loss due to various causes, including change in eating habits and increased internal and externals stress. Although genetic factors are known as the major cause of hair loss, increased social stress, environmental pollution, frequent perms and dyeing, wrong scalp management, etc. are also regarded as the cause of hair loss.

People have about 100,000-150,000 hairs, and each hair has a different growth cycle.

In the early stage of development, hair follicles are formed as follows. Placodes are formed by basal epidermal cells by the action of β-catenin (Sarah E. Miller, 2002). In response to signals from the placodes, dermal fibroblasts form dermal condensates. At the same time, the placodes grow and invaginate toward the dermis. They are combined with the dermal condensates and form primary hair germs. After the hair germs are formed, the epidermal cells grow elongatedly toward the dermis, forming hair pegs, which are multi-layered structures in the form of long pillars. At the same time, the dermal condensates form spherical dermal papillae. The hair pegs form hair bulbs as the end portion becomes gradually thicker. Hair follicles are formed as the hair bulbs surround the dermal papillae, and hair is produced as the epidermal cells in the hair follicles are differentiated.

The newly produced hair passes through an anagen stage, a catagen stage and a telogen stage. After the hair is lost, the anagen stage begins again.

The hair growth cycle consists of three stages: the anagen stage where the hair grows actively, the catagen stage where the degradation of hair begins, and the telogen (resting) stage where the hair growth is stopped.

It is known that the activity of dermal papilla cells constituting the hair follicles and outer root sheath cells including hair follicle stem cells and a variety of cytokines and growth factors produced by these cells play an important role in the regulation of the hair growth cycle. For periodic hair growth, the regeneration of hair follicles and the activation of stem cells are necessary, which are known to be accomplished by the production and activation of Wnt/β-catenin (Ito M, Yang Z, Andl T, Cui C, Kim N, Millar S E, and Cotsarelis G, Wnt-dependent de novo hair follicle regeneration in adult mouse skin after wounding. Nature, 2007, 447(7142): pp. 316-320). The reconstruction of hair follicles during the hair growth cycle from the telogen stage to the anagen stage is mediated by Wnt signals.

In general, hair loss refers to a condition wherein the number of abnormally shedding hairs increases as the proportion of hairs in the anagen stage is decreased and the proportion of hairs in the catagen or telogen stage is increased. Whereas normal people have many hairs in the anagen stage, those with hair loss (alopecia) show hair loss because they have many hairs in the telogen stage. People with hair loss show characteristic hair miniaturization. As hair loss proceeds, the duration of the anagen stage is decreased and the transition to the catagen and telogen stages is facilitated. Subsequently, the hair follicles are gradually miniaturized as the volume of dermal papillae is decreased. Therefore, for treatment of hair loss, it is important to facilitate the transition of hair follicles in the telogen stage to the anagen stage and increase the duration of the reduced anagen stage.

Although excessive male hormone production, excessive sebum secretion, scalp function deterioration caused by poor blood circulation, peroxides, bacteria, etc., genetic factors, aging, stress, etc. have been discussed as the causes of hair loss, the cause of hair loss not been elucidated clearly yet. Currently known products for preventing hair loss contain ingredients for promoting blood circulation, strengthening hair follicle functions, moisturizing the scalp, preventing dandruff, preventing oxidation, extending the anagen stage, inhibiting the action of male hormone, etc. as active ingredients. However, no distinct effect has been achieved yet and side effects often cause problems.

Currently developed agents for treating or preventing hair loss include those containing active ingredients for promoting blood circulation, strengthening hair follicle functions, or moisturizing the scalp; or female hormone for inhibiting male hormone. In addition, the above agents include minoxidil, finasteride, trichosaccharide, etc.

However, the commercially available products have no effect for hair loss that has progressed already or are limited in use because of side effects such as inhibition of male hormone or other problems.

### DISCLOSURE

### Technical Problem

The present disclosure is directed to providing a composition for preventing (alleviating) hair loss or promoting hair regrowth, which provides a superior effect of promoting hair regrowth and/or preventing hair loss while being safe for the human body and having no side effect.

In addition, the present disclosure is directed to providing a hair or scalp product, which contains the composition for preventing hair loss or promoting hair regrowth.

### Technical Solution

The inventors of the present disclosure have made consistent efforts to develop a composition that can act effectively while being safe for the human body without the disadvantage of the insignificant effect of preventing (alleviating) hair loss or promoting hair regrowth of the existing agent for treating hair loss.

As a result, they have found out that a composition containing one or more of valechlorine, tussilagone, periplocin, lysionotin, nitidine, lanosterol, engeletin, specnuezhenide, praeruptorin B, rotundic acid, sesamoside, polyphyllin VII or pharmaceutically or cosmetically acceptable salts thereof as an active ingredient has the effect of promoting hair regrowth and/or preventing hair loss, and have completed the present disclosure.

Specifically, the inventors of the present disclosure have identified that each of the above-described active ingredients promotes the activity of the Wnt/β-catenin signaling pathway, promotes the proliferation of dermal papilla cells, and increases the expression of β-catenin in dermal papilla cells. That is to say, they have confirmed through in-vitro experiments that each of the active ingredients play an important role in the transition from the catagen or telogen stage to the anagen stage in the hair growth cycle by promoting the activity of the Wnt/β-catenin signaling pathway, promoting the proliferation of dermal papilla cells and increasing the expression of β-catenin in dermal papilla cells.

Due to the above-described effects, it is expected that the composition for preventing hair loss and/or promoting hair regrowth of the present disclosure will promote hair regrowth by facilitating the transition from the telogen stage to the anagen stage in the hair growth cycle and prevent hair loss by delaying the transition to the catagen stag. However, the present disclosure is not limited by such a mechanism.

In addition, the inventors of the present disclosure have identified that a cosmetic composition containing the above-described active ingredients has a remarkably superior effect of improving hair thickness and density when it is treated to a patient with hair loss.

Additionally, they have identified through a comparative experiment with the commercially available minoxidil that a composition containing the active ingredients of the present disclosure have a remarkably superior effect of preventing hair loss and/or promoting hair regrowth as compared to minoxidil.

The present disclosure provides a composition for preventing hair loss or promoting hair regrowth, which contains one or more of valechlorine, tussilagone, periplocin, lysionotin, nitidine, lanosterol, engeletin, specnuezhenide, praeruptorin B, rotundic acid, sesamoside, polyphyllin VII or pharmaceutically or cosmetically acceptable salts thereof as an active ingredient.

The chemical formulas and structures of the twelve active ingredients are described in Table 1.

TABLE 1

| Active ingredients | Chemical formulas |
|---|---|
| Lysionotin | 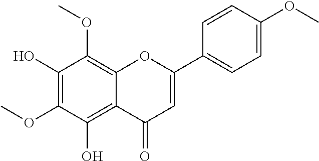 |
| Nitidine | 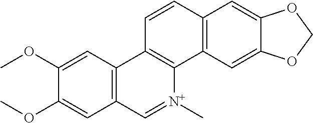 [SMILES: COc1cc2c[n+](C)c3c4cc5c(cc4ccc3c2cc1OC)OCO5] |
| Rotundic acid | 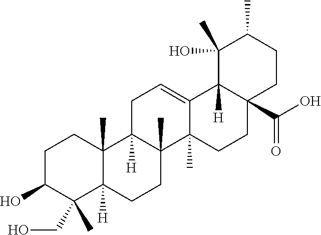 |
| Specnuezhenide | 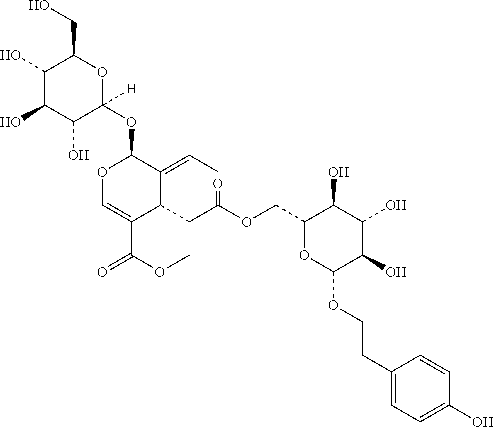 |
| Tussilagone | 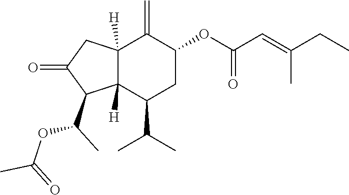 [SMILES: C=C1[C@H](OC(=O)/C=C(\C)CC)C[C@@H](C(C)C)[C@@H]2[C@@H]([C@H](C)OC(C)=O)C(=O)C[C@@H]12] |
| Valechlorine | 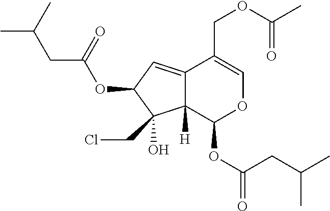 [SMILES: CC(=O)OCC1=CO[C@@H](OC(=O)CC(C)C)[C@H]2C1=C[C@H](OC(=O)CC(C)C)[C@]2(O)CCl] |
| Lanosterol | 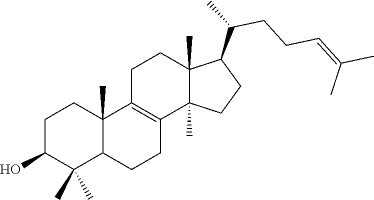 |
| Polyphyllin VII | 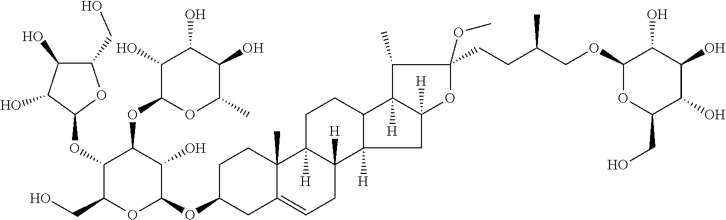 |
| Praeruptorin B | 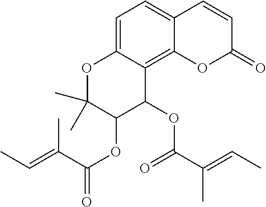 [SMILES: C/C=C(\C)C(=O)OC1c2c(ccc3ccc(=O)oc23)OC(C)(C)C1OC(=O)/C(C)=C/C] |
| Periplocin | 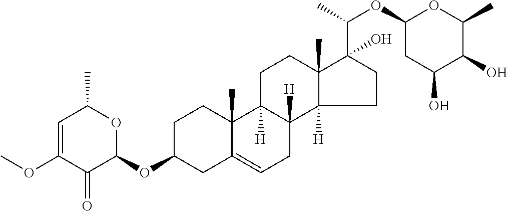 |
| Sesamoside | 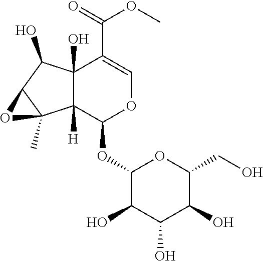 [SMILES: COC(=O)C1=CO[C@@H](O[C@@H]2O[C@H](CO)[C@@H](O)[C@H](O)[C@H]2O)[C@@H]2[C@@]3(C)O[C@H]3[C@@H](O)[C@]12O] |
| Engeletin | 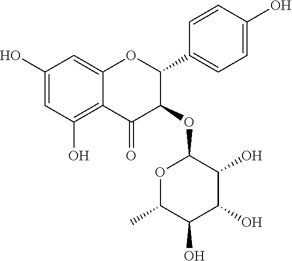 [SMILES: C[C@@H]1O[C@@H](O[C@H]2C(=O)c3c(O)cc(O)cc3O[C@@H]2c2ccc(O)cc2)[C@H](O)[C@H](O)[C@H]1O] |

In an exemplary embodiment, the twelve active ingredients may be derived from plants.

The valechlorine may be derived from valerian (Valeriana officinalis L.), the tussilagone may be derived from coltsfoot (Tussilago farfara), the periplocin may be derived from Periploca sepium Bge, the lysionotin may be derived from fewflower lysionotus herb, the nitidine may be derived from zanthoxylum (Zanthoxylum nitidum (Roxb.) DC.), the lanosterol may be derived from purslane (Portulaca oleracea L.), the engeletin may be derived from Engelhardtia roxburghiana Wall, the specnuezhenide may be derived from glossy privet (Ligustrum lucidum) fruit extract, the praeruptorin B may be derived from Peucedanum praeruptorum Dunn, the rotundic acid may be derived from Euscaphis japonica, the sesamoside may be derived from sesame (Sesamum angolense), and the polyphyllin VII may be derived from Paris polyphylla.

In an exemplary embodiment, one or more of the valechlorine, tussilagone, periplocin, lysionotin, nitidine, lanosterol, engeletin, specnuezhenide, praeruptorin B, rotundic acid, sesamoside, polyphyllin VII or pharmaceutically or cosmetically acceptable salts thereof may be contained in an amount of 0.00001-50 wt %, preferably 0.0001-20 wt %, more preferably 0.001-10 wt %, further more preferably 0.01-5 wt %, based on the total weight of the composition.

In addition, the present disclosure provides a pharmaceutical drug, a quasi-drug, a cosmetic or a functional health food, which contains the composition for preventing hair loss or promoting hair regrowth. The composition of the present disclosure may be formulated into any form that can be commonly applied to skin. Specifically, it may be formulated into a formulation for external application. For example, the composition of the present disclosure may be prepared into a formulation applicable to skin, such as a liquid, a cream, a paste, a solid, etc.

The one or more of the valechlorine, tussilagone, periplocin, lysionotin, nitidine, lanosterol, engeletin, specnuezhenide, praeruptorin B, rotundic acid, sesamoside, polyphyllin VII or pharmaceutically or cosmetically acceptable salts thereof of the present disclosure may be contained in the above-described composition or formulation as an agent for preventing hair loss and/or promoting hair regrowth.

In an exemplary embodiment, when the formulation of the present disclosure is a liquid, a solvent, a solubilizer, an emulsifier, etc. may be used as a carrier ingredient. For example, water, an alcohol, ethyl carbonate, ethyl acetate, benzyl alcohol, benzyl benzoate, propylene glycol, 1,3-butylene glycol, an aliphatic glycerol ester, a fatty acid ester of polyethylene glycol or sorbitan, etc. may be used as the carrier ingredient.

The alcohol may be specifically a linear or branched C₂-C₄ monoalcohol, more specifically ethanol or isopropanol. Particularly, when ethanol or isopropanol is used as the carrier ingredient, the transdermal absorption of the composition for preventing hair loss or promoting hair regrowth may be facilitated.

In an exemplary embodiment, when the formulation of the present disclosure is a paste, a cream or a gel, an animal fiber, a plant fiber, a wax, paraffin, starch, tragacanth, a cellulose derivative, polyethylene glycol, silicone, bentonite, silica, talc, zinc oxide, etc. may be used as a carrier ingredient.

In an exemplary embodiment, when the formulation of the present disclosure is a powder or a spray, lactose, talc, silica, aluminum hydroxide, calcium silicate, polyamide powder, etc. may be used as a carrier ingredient. Particularly, a spray formulation may further contain a propellant such as chlorofluorohydrocarbon, propane/butane, dimethyl ether, etc.

In an exemplary embodiment, the composition of the present disclosure may further contain ingredients commonly used in formulations for external application to skin. For example, one or more additive selected from a group consisting of water, a surfactant, a moisturizer, a lower alcohol, a chelating agent, a sterilizer, an antioxidant, an antiseptic, a colorant, a flavorant, etc. may be further contained.

The present disclosure provides a hair or scalp product containing the composition for preventing hair loss or promoting hair regrowth described above.

The hair or scalp product may be any one selected from a group consisting of a shampoo, a tonic, a conditioner, a lotion, a serum, an ampule, a treatment, a moose, a gel, a pack, a cream, an essence, a powder, a spray, an oil, a soap and an ointment, although not being limited thereto.

In addition, the hair or scalp product may be any one selected from a group consisting of a cleanser, a hairdye, a perm agent, a hairstyling agent, a scalp clinic agent, a hair clinic agent, a scalp scaling agent, a hair scaling agent, a scalp massage agent, a hair massage agent, a scalp care agent, a hair care agent or aerosol forms thereof, although not being limited thereto.

In an exemplary embodiment, the composition for preventing hair loss or promoting hair regrowth of the present disclosure may be formulated into a hair tonic or a hair lotion.

Specifically, the composition for preventing hair loss or promoting hair regrowth of the present disclosure may be administered transdermally by directly applying or spraying to skin.

The present disclosure also provides a functional health food composition containing the composition for preventing hair loss or promoting hair regrowth described above. The functional health food may be prepared by a method commonly used in the art. During the preparation, raw materials and ingredients commonly used in the art may be added. In addition, the functional health food may be prepared into any formulation acknowledged as a functional health food. The functional health food of the present disclosure may be ingested as a supplement for enhancing the effect of preventing hair loss or promoting hair regrowth since it can be prepared into various formulations, has advantages in that it is prepared from food raw materials and has no side effect that may occur during long-term intake of pharmaceutical drugs, and has excellent portability.

In addition, the present disclosure provides a method for preventing hair loss or promoting hair regrowth, which includes a step of administering a composition for preventing hair loss or promoting hair regrowth, containing one or more of valechlorine, tussilagone, periplocin, lysionotin, nitidine, lanosterol, engeletin, specnuezhenide, praeruptorin B, rotundic acid, sesamoside, polyphyllin VII or pharmaceutically or cosmetically acceptable salts thereof as an active ingredient, to a patient (subject).

The term “administration” used in the present disclosure may refer to introduction of the composition of the present disclosure by any suitable means. The composition of the present disclosure may be administered via any general route that can reach the desired tissue. Specifically, it may be administered transdermally or orally, more specifically transdermally, most specifically topically. The number of administration of the composition of the present disclosure may be determined by prescription or as necessary or desired.

The contents of all the ingredients described in the present disclosure do not exceed the highest limits specified in related regulations and standards of Korea, China, USA, Europe, Japan, etc. (e.g., Regulations on the Safety Standards, etc. of Cosmetics (Korea), Safety and Technical Standards for Cosmetics (China), Food Code (Korea), Food Additives Code (Korea), Functional Health Food Code (Korea), Standards for Sanitation (China), etc.). That is to say, the cosmetic, food or personal care composition according to the present disclosure contains the ingredients according to the present disclosure within the content limits approved by the regulations and standards of various countries.

The inventors of the present disclosure have confirmed through in-vitro experiments that the one or more of the valechlorine, tussilagone, periplocin, lysionotin, nitidine, lanosterol, engeletin, specnuezhenide, praeruptorin B, rotundic acid, sesamoside, polyphyllin VII or pharmaceutically or cosmetically acceptable salts thereof contained in the composition of the present disclosure plays an important role in the transition from the catagen or telogen stage to the anagen stage in the hair growth cycle by amplifying Wnt/β-catenin signaling by dermal papilla cells (Experimental Examples 1, 2 and 3).

The amount of the composition of the present disclosure may be controlled adequately depending on the age or condition of a subject or the particular formulation. Specifically, it may be applied to the scalp once or several times a day, for a week to a few months. In Experimental Example of the present disclosure, the composition I for preventing hair loss or and/or promoting hair regrowth (Preparation Example 1) showed superior effect of promoting hair regrowth when it was used 5 times a week for 6 months (Experimental Example 4).

### Advantageous Effects

A composition for preventing (alleviating) hair loss and/or promoting hair regrowth of the present disclosure exhibits excellent effect of promoting hair regrowth and superior effect of preventing/delaying hair loss.

### DESCRIPTION OF DRAWINGS

FIG. 1 shows a result of measuring the activation of the Wnt/β-catenin promoter by lysionotin, nitidine, rotundic acid or specnuezhenide.

FIG. 2 shows a result of measuring the activation of the Wnt/β-catenin promoter by tussilagone, valechlorine, lanosterol or polyphyllin VII.

FIG. 3 shows a result of measuring the activation of the Wnt/β-catenin promoter by praeruptorin B, periplocin, sesamoside or engeletin.

### BEST MODE

Hereinafter, the present disclosure will be described in detail through examples. However, the examples of the present disclosure can be changed into various other forms, and the scope of the present disclosure is not limited by the examples. The examples of the present disclosure are provided so that the present disclosure is described more completely understood by those having ordinary knowledge in the art.

### Preparation Example 1: Composition I for Treating Hair Loss and/or Promoting Hair Regrowth (Hair Tonic)

Hair tonic compositions containing valechlorine, tussilagone, periplocin, lysionotin, nitidine, lanosterol, engeletin, specnuezhenide, praeruptorin B, rotundic acid, sesamoside or polyphyllin VII as an active ingredient were prepared as Examples 1 to 12-2 as described in Table 2.

The materials and reagents used in the experiments were commercially purchased from manufacturers and supplies of cosmetic raw materials.

TABLE 2

| | | | | | | Flavorant | |
|---|---|---|---|---|---|---|---|
| Ingredients | | Castor | | | | and | Purified |
| (unit: wt %) | Ethanol | oil | Glycerin | Active ingredient | | colorant | water |
| Comp. Ex. 1 | 55 | 5 | 3 | — | | Adequate | Balance |
| | | | | | | | (to 100) |
| Comp. Ex. 2 | 55 | 5 | 3 | Minoxidil | 2 μg/mL | Adequate | Balance |
| | | | | | | | (to 100) |
| Comp. Ex. 3 | 0 | 5 | 3 | — | | Adequate | Balance |
| | | | | | | | (to 100) |
| Ex. 1 | 55 | 5 | 3 | Valechlorine | 100 μg/mL | Adequate | Balance |
| | | | | | | | (to 100) |
| Ex. 1-2 | 0 | 5 | 3 | Valechlorine | 100 μg/mL | Adequate | Balance |
| | | | | | | | (to 100) |
| Ex. 2 | 55 | 5 | 3 | Tussilagone | 100 μg/mL | Adequate | Balance |
| | | | | | | | (to 100) |
| Ex. 2-2 | 0 | 5 | 3 | Tussilagone | 100 μg/mL | Adequate | Balance |
| | | | | | | | (to 100) |
| Ex. 3 | 55 | 5 | 3 | Periplocin | 100 μg/mL | Adequate | Balance |
| | | | | | | | (to 100) |
| Ex. 3-2 | 0 | 5 | 3 | Periplocin | 100 μg/mL | Adequate | Balance |
| | | | | | | | (to 100) |
| Ex. 4 | 55 | 5 | 3 | Lysionotin | 100 μg/mL | Adequate | Balance |
| | | | | | | | (to 100) |
| Ex. 4-2 | 0 | 5 | 3 | Lysionotin | 100 μg/mL | Adequate | Balance |
| | | | | | | | (to 100) |
| Ex. 5 | 55 | 5 | 3 | Nitidine | 100 μg/mL | Adequate | Balance |
| | | | | | | | (to 100) |
| Ex. 5-2 | 0 | 5 | 3 | Nitidine | 100 μg/mL | Adequate | Balance |
| | | | | | | | (to 100) |
| Ex. 6 | 55 | 5 | 3 | Lanosterol | 100 μg/mL | Adequate | Balance |
| | | | | | | | (to 100) |
| Ex. 6-2 | 0 | 5 | 3 | Lanosterol | 100 μg/mL | Adequate | Balance |
| | | | | | | | (to 100) |
| Ex. 7 | 55 | 5 | 3 | Engeletin | 100 μg/mL | Adequate | Balance |
| | | | | | | | (to 100) |
| Ex. 7-2 | 0 | 5 | 3 | Engeletin | 100 μg/mL | Adequate | Balance |
| | | | | | | | (to 100) |
| Ex. 8 | 55 | 5 | 3 | Specnuezhenide | 100 μg/mL | Adequate | Balance |
| | | | | | | | (to 100) |
| Ex. 8-2 | 0 | 5 | 3 | Specnuezhenide | 100 μg/mL | Adequate | Balance |
| | | | | | | | (to 100) |
| Ex. 9 | 55 | 5 | 3 | Praeruptorin B | 100 μg/mL | Adequate | Balance |
| | | | | | | | (to 100) |
| Ex. 9-2 | 0 | 5 | 3 | Praeruptorin B | 100 μg/mL | Adequate | Balance |
| | | | | | | | (to 100) |
| Ex. 10 | 55 | 5 | 3 | Rotundic acid | 100 μg/mL | Adequate | Balance |
| | | | | | | | (to 100) |
| Ex. 10-2 | 0 | 5 | 3 | Rotundic acid | 100 μg/mL | Adequate | Balance |
| | | | | | | | (to 100) |
| Ex. 11 | 55 | 5 | 3 | Sesamoside | 100 μg/mL | Adequate | Balance |
| | | | | | | | (to 100) |
| Ex. 11-2 | 0 | 5 | 3 | Sesamoside | 100 μg/mL | Adequate | Balance |
| | | | | | | | (to 100) |
| Ex. 12 | 55 | 5 | 3 | Polyphyllin VII | 100 μg/mL | Adequate | Balance |
| | | | | | | | (to 100) |
| Ex. 12-2 | 0 | 5 | 3 | Polyphyllin VII | 100 μg/mL | Adequate | Balance |
| | | | | | | | (to 100) |

### Preparation Example 2: Composition II for Treating Hair Loss and/or Promoting Hair Regrowth (Hair Lotion)

Hair lotion compositions containing valechlorine, tussilagone, periplocin, lysionotin, nitidine, lanosterol, engeletin, specnuezhenide, praeruptorin B, rotundic acid, sesamoside or polyphyllin VII as an active ingredient were prepared as described in Table 2.

TABLE 3

| Ingredients | Weight ratio (%) |
|---|---|
| Cetostearyl alcohol | 2 |
| Stearyltriethylammonium chloride | 2 |
| Hydroxyethyl cellulose | 0.5 |
| Valechlorine, tussilagone, periplocin, | 0.01 |
| lysionotin, nitidine, lanosterol, engeletin, | |
| specnuezhenide, praeruptorin B, rotundic | |
| acid, sesamoside or polyphyllin VII | |
| Flavorant and colorant | Adequate |
| Purified water | Balance (to 100) |

Experimental Example 1: Regulation of Wnt/β-Catenin Signaling in Dermal Papilla Cells

In general, the activation of Wnt/β-catenin signaling in dermal papillae occurs during the transition from the catagen or telogen stage to the anagen stage in the hair growth cycle, i.e., throughout the anagen stage where hair growth occurs. In addition, if the Wnt/β-catenin signaling is weakened or stopped in the telogen or catagen stage, hair follicles are degenerated and hair loss occurs.

Therefore, it was investigated how much the valechlorine, tussilagone, periplocin, lysionotin, nitidine, lanosterol, engeletin, specnuezhenide, praeruptorin B, rotundic acid, sesamoside or polyphyllin VII of the present disclosure contributes to the amplification of the Wnt/β-catenin signaling.

DMSO was used as a negative control group.

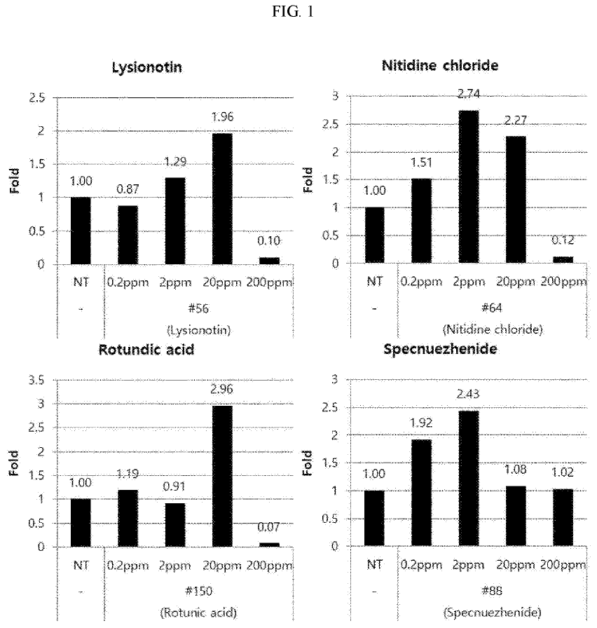

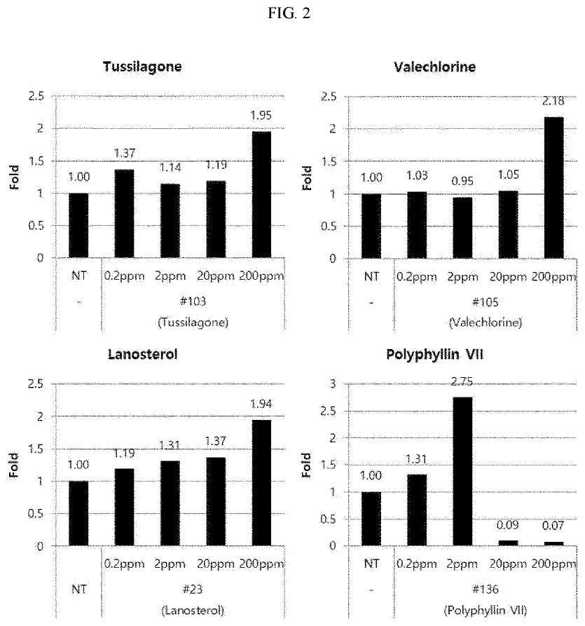

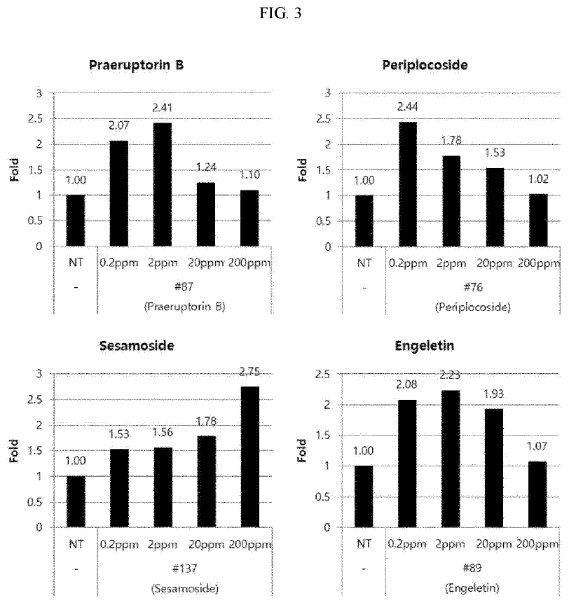

After seeding 3×10⁴ Wnt reporter HEK293A cells onto each well of a 96-well plate, they were treated with the valechlorine, tussilagone, periplocin, lysionotin, nitidine, lanosterol, engeletin, specnuezhenide, praeruptorin B, rotundic acid, sesamoside or polyphyllin VII of the present disclosure at concentrations of 200 μg/mL, 20 μg/mL, 2 μg/mL or 0.2 μg/mL. Then, reporter assay was conducted using a Luciferase assay kit (E1960, Promega) according to the manufacturer's protocol. The activity of the Wnt/β-catenin promoter was measured using a luminometer Victor (PerkinElmer, Waltham, Massachusetts, USA). The result is shown in FIG. 1, FIG. 2 and FIG. 3.

As a result, the Wnt/β-catenin signaling was amplified about 2-fold or higher in the groups treated with valechlorine, tussilagone, periplocin, lysionotin, nitidine, lanosterol, engeletin, specnuezhenide, praeruptorin B, rotundic acid, sesamoside or polyphyllin VII as compared to the untreated negative control group.

Accordingly, it was confirmed that the valechlorine, tussilagone, periplocin, lysionotin, nitidine, lanosterol, engeletin, specnuezhenide, praeruptorin B, rotundic acid, sesamoside or polyphyllin VII is effective in promoting hair regrowth because it promotes the activity of the Wnt/β-catenin signaling pathway, associated with promotion of hair growth by hair follicle stem cells, very superiorly.

### Experimental Example 2: Proliferation of Dermal Papilla Cells

The effect of promoting the proliferation of dermal papilla cells was investigated using human-derived dermal papilla cells (DPCs). The human-derived dermal papilla cells were purchased from PromoCell. The DPCs were cultured using a medium (follicle dermal papilla cell growth medium; PromoCell, Heidelberg, Germany) and a medium additive (supplement mix; PromoCell, Heidelberg, Germany) recommended by PromoCell under the condition of 37° C. and 5% CO₂. The cultured DPCs were seeded onto a 96-well plate at 3,000 cells/well and then cultured for 24 hours under 0.1% serum condition. Then, the cultured cells were incubated for 1 day after treating with DMSO diluted with serum-free DMEM to 1:1000 (vehicle), 0.1 μg/mL minoxidil as a positive control group, or at 2 or 20 μg/mL valechlorine, tussilagone, periplocin, lysionotin, nitidine, lanosterol, engeletin, specnuezhenide, praeruptorin B, rotundic acid, sesamoside or polyphyllin VII. After the incubation, cell proliferation was evaluated by the CCK method. CCK-8 was treated to a medium at 1:10 and then incubated for 1 hour. 1 hour later, the absorbance of each well was measured at 450 nm. All experiments were repeated 3 times and the average of absorbance was calculated. The control group was set at 100%, and the results were represented as a percentage compared to the control group.

As a result, the valechlorine, tussilagone, periplocin, lysionotin, nitidine, lanosterol, engeletin, specnuezhenide, praeruptorin B, rotundic acid, sesamoside or polyphyllin VII showed superior effect of promoting the proliferation of dermal papilla cells (Table 4).

TABLE 4

| | | |
|---|---|---|
| | | Proliferation of dermal |
| Groups | Concentration | papilla cells (%) |

| | | |
|---|---|---|
| Untreated | — | 100 |

| | | | |
|---|---|---|---|
| Minoxidil | 100 | ng/ml | 130.1 |
| Valechlorine | 2 | μg/mL | 146.4 |
| | 20 | μg/mL | 159.4 |
| Tussilagone | 2 | μg/mL | 156.8 |
| | 20 | μg/mL | 178.3 |
| Periplocin | 2 | μg/mL | 144.4 |
| | 20 | μg/mL | 173.1 |
| Lysionotin | 2 | μg/mL | 164.3 |
| | 20 | μg/mL | 179.1 |
| Nitidine | 2 | μg/mL | 155.2 |
| | 20 | μg/mL | 180.6 |
| Lanosterol | 2 | μg/mL | 142.7 |
| | 20 | μg/mL | 188.9 |
| Engeletin | 2 | μg/mL | 143.2 |
| | 20 | μg/mL | 188.1 |
| Specnuezhenide | 2 | μg/mL | 150.3 |
| | 20 | μg/mL | 169.7 |
| Praeruptorin B | 2 | μg/mL | 144.2 |
| | 20 | μg/mL | 173.2 |
| Rotundic acid | 2 | μg/mL | 156.3 |
| | 20 | μg/mL | 183.4 |
| Sesamoside | 2 | μg/mL | 167.6 |
| | 20 | μg/mL | 193.5 |
| Polyphyllin VII | 2 | μg/mL | 144.5 |
| | 20 | μg/mL | 182.4 |

### Experimental Example 3: Increased Expression of β-Catenin in Dermal Papilla Cells

During the telogen or catagen stage when Wnt signaling is inhibited, β-catenin is degraded by GSK3-beta in dermal papilla cells. But, during the anagen stage when the Wnt signaling is amplified, the expression level of β-catenin is increased in the dermal papilla cells because the activity of GSK3-beta is inhibited.

Human dermal papilla cells were seeded onto a 6-well plate at 500,000 cells/well and then incubated for 1 day after treating with 2 or 20 μg/mL valechlorine, tussilagone, periplocin, lysionotin, nitidine, lanosterol, engeletin, specnuezhenide, praeruptorin B, rotundic acid, sesamoside or polyphyllin VII. After the incubation, the cells were lysed using an M-PER lysis buffer and a cell lysate of the same volume was loaded on SDS-PAGE gel. After transferring the cell lysate loaded on the SDS-PAGE gel to a nitrocellulose membrane, proteins were detected by attaching anti-β-catenin antibodies to the nitrocellulose membrane. GAPDH was used as a control for calibrating the quantity of proteins. The amount of β-catenin increased by the valechlorine, tussilagone, periplocin, lysionotin, nitidine, lanosterol, engeletin, specnuezhenide, praeruptorin B, rotundic acid, sesamoside or polyphyllin VII was investigated. The control group was set at 100%, and the results were represented as a percentage compared to the control group as shown in Table 5.

TABLE 5

| | | |
|---|---|---|
| | | Expression level of |
| Groups | Concentration | β-catenin protein (%) |

| | | |
|---|---|---|
| Untreated | — | 100 |

| | | | |
|---|---|---|---|
| Wnt3a | 0.5 | μg/mL | 208.8 |
| Valechlorine | 2 | μg/mL | 216.6 |
| | 20 | μg/mL | 233.6 |
| Tussilagone | 2 | μg/mL | 208.2 |
| | 20 | μg/mL | 253.2 |
| Periplocin | 2 | μg/mL | 218.5 |
| | 20 | μg/mL | 267.3 |
| Lysionotin | 2 | μg/mL | 210.0 |
| | 20 | μg/mL | 231.5 |
| Nitidine | 2 | μg/mL | 219.8 |
| | 20 | μg/mL | 261.7 |
| Lanosterol | 2 | μg/mL | 240.6 |
| | 20 | μg/mL | 245.5 |
| Engeletin | 2 | μg/mL | 231.1 |
| | 20 | μg/mL | 276.3 |
| Specnuezhenide | 2 | μg/mL | 215.8 |
| | 20 | μg/mL | 289.4 |
| Praeruptorin B | 2 | μg/mL | 210.7 |
| | 20 | μg/mL | 250.4 |
| Rotundic acid | 2 | μg/mL | 211.1 |
| | 20 | μg/mL | 257.5 |
| Sesamoside | 2 | μg/mL | 245.4 |
| | 20 | μg/mL | 295.4 |
| Polyphyllin VII | 2 | μg/mL | 210.1 |
| | 20 | μg/mL | 254.6 |

As a result, it was confirmed that the amount of β-catenin was increased significantly in the groups treated with valechlorine, tussilagone, periplocin, lysionotin, nitidine, lanosterol, engeletin, specnuezhenide, praeruptorin B, rotundic acid, sesamoside or polyphyllin VII as compared to the untreated group, suggesting that Wnt/β-catenin signaling was increased in the dermal papilla cells. In particular, it was confirmed that a better effect was shown as compared to Wnt3a protein (positive control group) which binds directly to the receptor of Wnt/β-catenin (In the industry, an increase of 1.5-fold or higher is generally regarded as being effective for promoting hair regrowth.)

### Experimental Example 4: Hair Growth-Promoting Effect of Composition I for Treating Hair Loss or Promoting Hair Regrowth (Hair Tonic)

The hair growth-promoting effect of the composition I for treating hair loss or promoting hair regrowth (hair tonic) of the present disclosure was tested for 135 men and women having significantly less hairs or weak hairs with hair loss symptoms as compared to normal people. Different compositions were applied on the left and right parts of the scalp of the 135 men and women. The compositions of Comparative Examples 1-3 and Examples 1 to 12-2 were applied to 27 groups of 10 people, 5 times a week for 6 months. Evaluation was conducted using clinical images and phototrichograms. The evaluation using clinical images was conducted 4 months and 6 months after the application as ‘good’, ‘slight’ or ‘no change’ (good: improved by 50-75%, slight: improved by 25-50%, no change: no change). The evaluation of the number of hairs per unit area and average hair thickness using phototrichograms was conducted 4 months and 6 months after the application. The results are shown in Table 6.

TABLE 6

| | Evaluation using clinical images | | | | | | Evaluation using phototrichograms | | | | | |
|---|---|---|---|---|---|---|---|---|---|---|---|---|
| | | | | | | | Number of hairs per unit | | | Average hair thickness | | |
| | No change | | Slight | | Good | | area (/cm²) | | | (μm) | | |
| Sample | 4 m | 6 m | 4 m | 6 m | 4 m | 6 m | 0 m | 4 m | 6 m | 0 m | 4 m | 6 m |
| Comp. | 8 | 7 | 2 | 2 | 0 | 1 | 142 ± 21 | 146 ± 15 | 148 ± 15 | 53 ± 8 | 55 ± 4 | 58 ± 6 |
| Ex. 1 | | | | | | | | | | | | |
| Comp. | 6 | 4 | 3 | 2 | 1 | 4 | 138 ± 10 | 145 ± 11 | 160 ± 31 | 54 ± 5 | 58 ± 5 | 62 ± 6 |
| Ex. 2 | | | | | | | | | | | | |
| Comp. | 10 | 9 | 0 | 1 | 0 | 0 | 137 ± 15 | 142 ± 12 | 141 ± 33 | 49 ± 5 | 49 ± 4 | 52 ± 3 |
| Ex. 3 | | | | | | | | | | | | |
| Ex. 1 | 4 | 2 | 4 | 4 | 2 | 4 | 144 ± 17 | 152 ± 9 | 169 ± 15 | 41 ± 10 | 68 ± 1 | 73 ± 1 |
| Ex. 1-2 | 4 | 2 | 4 | 4 | 2 | 4 | 139 ± 23 | 146 ± 18 | 160 ± 12 | 49 ± 5 | 66 ± 5 | 69 ± 5 |
| Ex. 2 | 3 | 2 | 6 | 3 | 1 | 5 | 130 ± 19 | 155 ± 11 | 173 ± 21 | 55 ± 7 | 68 ± 6 | 77 ± 7 |
| Ex. 2-2 | 3 | 2 | 5 | 4 | 2 | 4 | 131 ± 25 | 150 ± 15 | 169 ± 11 | 41 ± 8 | 62 ± 2 | 70 ± 4 |
| Ex. 3 | 4 | 1 | 5 | 3 | 1 | 6 | 143 ± 13 | 165 ± 13 | 177 ± 20 | 48 ± 5 | 69 ± 9 | 79 ± 6 |
| Ex. 3-2 | 4 | 2 | 4 | 2 | 2 | 6 | 146 ± 10 | 160 ± 11 | 170 ± 08 | 45 ± 3 | 62 ± 5 | 73 ± 5 |
| Ex. 4 | 2 | 1 | 5 | 4 | 3 | 5 | 133 ± 18 | 155 ± 13 | 169 ± 11 | 43 ± 9 | 68 ± 7 | 76 ± 6 |
| Ex. 4-2 | 3 | 2 | 5 | 4 | 2 | 4 | 130 ± 11 | 150 ± 8 | 160 ± 10 | 40 ± 8 | 60 ± 6 | 70 ± 4 |
| Ex. 5 | 3 | 3 | 5 | 1 | 2 | 6 | 132 ± 16 | 156 ± 10 | 172 ± 13 | 43 ± 6 | 66 ± 7 | 73 ± 6 |
| Ex. 5-2 | 4 | 3 | 5 | 2 | 1 | 5 | 136 ± 20 | 150 ± 11 | 165 ± 7 | 42 ± 7 | 60 ± 9 | 69 ± 3 |
| Ex. 6 | 6 | 2 | 3 | 3 | 1 | 5 | 135 ± 15 | 153 ± 22 | 178 ± 21 | 51 ± 2 | 73 ± 7 | 83 ± 6 |
| Ex. 6-2 | 5 | 2 | 1 | 2 | 4 | 6 | 132 ± 25 | 145 ± 13 | 169 ± 10 | 49 ± 4 | 65 ± 5 | 75 ± 3 |
| Ex. 7 | 2 | 1 | 6 | 4 | 2 | 5 | 133 ± 22 | 157 ± 11 | 179 ± 11 | 53 ± 5 | 70 ± 8 | 88 ± 6 |
| Ex. 7-2 | 2 | 2 | 4 | 3 | 4 | 5 | 146 ± 13 | 151 ± 21 | 172 ± 20 | 51 ± 4 | 63 ± 6 | 73 ± 4 |
| Ex. 8 | 1 | 1 | 8 | 3 | 1 | 6 | 128 ± 20 | 168 ± 30 | 181 ± 16 | 48 ± 4 | 69 ± 6 | 79 ± 7 |
| Ex. 8-2 | 1 | 0 | 8 | 4 | 1 | 6 | 138 ± 13 | 164 ± 17 | 170 ± 11 | 43 ± 6 | 62 ± 5 | 72 ± 2 |
| Ex. 9 | 5 | 1 | 4 | 4 | 1 | 5 | 141 ± 18 | 177 ± 12 | 184 ± 14 | 42 ± 7 | 73 ± 7 | 83 ± 6 |
| Ex. 9-2 | 1 | 1 | 6 | 3 | 3 | 6 | 122 ± 25 | 149 ± 13 | 168 ± 16 | 55 ± 3 | 67 ± 4 | 77 ± 6 |
| Ex. 10 | 2 | 1 | 5 | 1 | 3 | 8 | 126 ± 16 | 159 ± 15 | 183 ± 11 | 52 ± 7 | 67 ± 6 | 88 ± 6 |
| Ex. 10-2 | 1 | 1 | 5 | 3 | 4 | 6 | 153 ± 10 | 172 ± 15 | 199 ± 13 | 49 ± 5 | 60 ± 6 | 73 ± 2 |
| Ex. 11 | 3 | 0 | 6 | 1 | 1 | 9 | 138 ± 11 | 174 ± 11 | 203 ± 21 | 45 ± 3 | 79 ± 6 | 89 ± 4 |
| Ex. 11-2 | 3 | 1 | 4 | 2 | 3 | 7 | 142 ± 13 | 169 ± 19 | 186 ± 11 | 49 ± 9 | 70 ± 4 | 81 ± 5 |
| Ex. 12 | 2 | 1 | 5 | 3 | 3 | 6 | 126 ± 22 | 149 ± 9 | 170 ± 12 | 51 ± 5 | 68 ± 4 | 79 ± 9 |
| Ex. 12-2 | 2 | 2 | 4 | 3 | 4 | 5 | 144 ± 13 | 154 ± 11 | 162 ± 13 | 45 ± 8 | 63 ± 6 | 72 ± 8 |

As seen from the above results, Examples 1 to 12-2 containing the valechlorine, tussilagone, periplocin, lysionotin, nitidine, lanosterol, engeletin, specnuezhenide, praeruptorin B, rotundic acid, sesamoside or polyphyllin VII showed high effect of preventing (alleviating) hair loss and promoting hair growth.

Through this, it was verified that the composition for preventing hair loss or promoting hair regrowth of the present disclosure containing the valechlorine, tussilagone, periplocin, lysionotin, nitidine, lanosterol, engeletin, specnuezhenide, praeruptorin B, rotundic acid, sesamoside or polyphyllin VII as an active ingredient can be very effectively used to treat hair loss.

It will be obvious to those having ordinary knowledge in the art that the formulation examples such as a hair tonic or a hair lotion are only examples of the composition for promoting hair regrowth of the present disclosure and the scope of the present disclosure composition is not limited by them.

After confirming that the valechlorine, tussilagone, periplocin, lysionotin, nitidine, lanosterol, engeletin, specnuezhenide, praeruptorin B, rotundic acid, sesamoside or polyphyllin VII has superior effect of promoting hair regrowth and preventing hair loss, the composition for preventing hair loss or promoting hair regrowth of the present disclosure was prepared into various formulations. Although a hair tonic and a hair lotion were described above as formulation examples, it will be obvious to those having ordinary knowledge in the art that the formulation examples are only examples of the composition of the present disclosure and the scope of the present disclosure composition is not limited by them.

In addition, a shampoo composition and a conditioner composition are described as examples in Table 7 and Table 8, respectively.

TABLE 7

| Ingredients | Contents (wt %) |
|---|---|
| Active ingredient for preventing hair loss | 0.01 |
| or promoting hair regrowth (one or more of | |
| valechlorine, tussilagone, periplocin, | |
| lysionotin, nitidine, lanosterol, engeletin, | |
| specnuezhenide, praeruptorin B, rotundic acid, | |
| sesamoside, polyphyllin VII or pharmaceutically | |
| or cosmetically acceptable salts thereof) | |
| Polyquaternium -10 | 0.5 |
| Sodium lauryl sulfate | 10 |
| Oil | 1 |
| Thickener | 5 |
| Flavorant | 0.5 |
| Sodium chloride | Adequate |
| Citric acid | Adequate |
| Water | Balance (to 100) |

TABLE 8

| Ingredients | Contents (wt %) |
|---|---|
| Active ingredient for preventing hair loss | 0.01 |
| or promoting hair regrowth (one or more of | |
| valechlorine, tussilagone, periplocin, | |
| lysionotin, nitidine, lanosterol, engeletin, | |
| specnuezhenide, praeruptorin B, rotundic acid, | |
| sesamoside, polyphyllin VII or pharmaceutically | |
| or cosmetically acceptable salts thereof) | |
| Stearamidopropyl diethylamine | 2 |
| Dicetyldiethylammonium chloride | 1 |
| Cetyl alcohol | 3 |
| Stearyl alcohol | 4 |
| Cyclomethicone | 5 |
| Flavorant | Adequate |
| Water | Balance (to 100) |

1. A composition for alleviating hair loss or promoting hair regrowth, comprising one or more of periplocin, tussilagone, nitidine, lysionotin, valechlorine, engeletin, specnuezhenide, praeruptorin B, rotundic acid, sesamoside, polyphyllin VII or pharmaceutically or cosmetically acceptable salts thereof as an active ingredient.

2. The composition for alleviating hair loss or promoting hair regrowth according to claim 1, wherein the composition promotes the activity of the Wnt/β-catenin signaling pathway.

3. The composition for alleviating hair loss or promoting hair regrowth according to claim 1, wherein the composition promotes the proliferation of dermal papilla cells.

4. The composition for alleviating hair loss or promoting hair regrowth according to claim 1, wherein the composition increases the expression of β-catenin in dermal papilla cells.

5. The composition for alleviating hair loss or promoting hair regrowth according to claim 1, wherein the composition is for a pharmaceutical drug, a quasi-drug, a cosmetic or a functional health food.

6. A hair or scalp product comprising the composition for alleviating hair loss or promoting hair regrowth according to claim 1.

7. The hair or scalp product according to claim 6, which is selected from a group consisting of a shampoo, a tonic, a conditioner, a lotion, a serum, an ampule, a treatment, a moose, a gel, a pack, a cream, an essence, a powder, a spray, an oil, a soap and an ointment.

8. The hair or scalp product according to claim 6, which is selected from a group consisting of a cleanser, a hairdye, a perm agent, a hairstyling agent, a scalp clinic agent, a hair clinic agent, a scalp scaling agent, a hair scaling agent, a scalp massage agent, a hair massage agent, a scalp care agent and a hair care agent.

9. A method for alleviating hair loss, or promoting hair regrowth comprising applying to a subject in need thereof a composition comprising one or more active ingredient selected from the group consisting of periplocin, tussilagone, nitidine, lysionotin, valechlorine, engeletin, specnuezhenide, praeruptorin B, rotundic acid, sesamoside, polyphyllin VII, and a pharmaceutically or cosmetically acceptable salt thereof.
